(19) United States
(12) Patent Application Publication
(10) Pub. No.: US 2026/0174464 A1
(43) Pub. Date: Jun. 25, 2026

(54) CHEST TUBE INSERTER FOR CHEST TUBE INSERTION AND DRUG INJECTION, AND KIT INCLUDING SAME

(71) Applicant: SEOUL NATIONAL UNIVERSITY HOSPITAL, Seoul (KR)

(72) Inventors: Woo Hyun JUNG, Seoul (KR); Suk Ki CHO, Seongnam-si, Gyeonggido (KR); Beatrice Chia Hui SHIH, Seoul (KR)

(73) Assignee: SEOUL NATIONAL UNIVERSITY HOSPITAL, Seoul (KR)

(21) Appl. No.: 19/123,848

(22) PCT Filed: Sep. 25, 2023
(86) PCT No.: PCT/KR2023/014701

§ 371 Date: Apr. 24, 2025

(30) Foreign Application Priority Data
Oct. 26, 2022 (KR) 10-2022-0138936

(51) Int. Cl. A61B 17/34; A61B 17/00; A61B 17/04; A61B 17/06; A61M 25/02

(52) U.S. Cl. CPC A61B 17/3468; A61B 17/0057; A61B 17/0485; A61M 25/02; A61B 2017/0458; A61B 2017/0464; A61B 2017/047; A61B 2017/0608; A61B 2017/3427; A61B 2017/3482

(57) ABSTRACT

The present invention relates to a chest tube inserter for chest tube insertion and drug injection, and a kit including same. To this end, the chest tube inserter according to the present invention passes through a chest wall, and in order to insert a chest tube, an insert guide hole is formed to penetrate a body portion of the chest tube inserter, so that the chest tube can be inserted. Accordingly, the chest tube inserter has the advantage of being able to easily insert the chest tube without causing pain when inserting the chest tube through the chest wall and also has the advantage of facilitating drug injection due to the body portion being formed in a double skin structure.

### TECHNICAL FIELD

The present disclosure relates to a chest tube inserter, which facilitates the insertion of a chest tube and allows for the injection of a drug for pain control at the insertion site of the chest tube, and a kit including the same, and more particularly to, a chest tube inserter for chest tube insertion and drug injection, which is provided to penetrate a chest wall and allows a chest tube passing through the inside of a body part to be easily inserted without pain, has a drug injection part connected thereto for injecting a drug for pain control, and allows the drug to be easily delivered to the chest wall into which a kit is inserted, and a kit including the same.

### BACKGROUND

Chest tube insertion, which involves inserting a chest tube into the chest cavity, is one of the most widely performed procedures. The procedure is performed for the purpose of removing air, blood, or pus when the former occurs inside the chest wall.

Conventional chest tube insertion proceeded in the manner of incising the chest wall to a certain size, inserting the end of a chest tube into the incision site while gripping it with forceps, and then pushing the chest tube into the chest cavity while holding the incision site open with the forceps.

However, there are problems that the muscles that make up the chest wall will contract again even if they are torn once, and when holding and inserting the forceps while gripping the chest tube, other muscle layers are torn, causing the patient to feel severe pain. In addition, the precision or accuracy of chest tube insertion varies depending on the skill level of the doctor even in relatively simple procedures, and there are instances where serious complications such as massive bleeding occur.

To solve these problems, chest tubes combined with dilators that widen the incision site so as to facilitate insertion when inserting the chest tube have recently been used. However, current dilators are limited in their applicability since they are dependent on the diameter of chest tubes, and doctors still have no choice but to incise the chest wall and insert a chest tube at their discretion.

Further, as a prior art document proposed to conduct the foregoing chest tube insertion more easily, there is Korean Patent Application Publication No. 10-2022-0057048 (published on May 9, 2022, hereinafter referred to as “prior art document”). The prior art document discloses an air injection stopper for a chest catheter that is fixed to the chest wall so that the chest catheter can be inserted stably into a surgical hole formed to penetrate the patient's chest cavity.

The air injection stopper for a chest catheter in the prior art document can perform the function of guiding the catheter to be easily inserted, but since the main object is to prevent air from leaking into the gap between the catheter and the chest wall in the process of injecting air, which would in turn reduce the air intake efficiency, the initial incision of the chest wall must be made arbitrarily by the doctor, and there is a limitation in that there is a lack of consideration for inflammation occurring at the incision site or pain felt by the patient.

In addition, there are cases where the inserted chest tube is left in place for a long time, and in such cases, the chest tube continuously irritates the intercostal nerves that run between the ribs, causing the patients to constantly feel excruciating pain, which has led to a need for a solution therefor.

### Technical Objects

The present disclosure has been designed to solve the problems described above, and it is an object to provide a chest tube inserter for chest tube insertion and drug injection, which can reduce the patient's pain by stably inserting a chest tube into the chest cavity while minimizing the incision of the chest wall, can continuously supply drugs so as to control pain caused by the placement of the chest tube at the incision site, and can stably fix the chest tube so as to prevent it from moving, and a kit including the same.

### Technical Solution

In order to achieve the above object, a chest tube inserter 100 for inserting a chest tube and injecting a drug according to one embodiment of the present disclosure is provided to penetrate a chest wall and is for inserting a chest tube, wherein a body part 110 of the chest tube inserter 100 has an insert guide hole 120 formed therethrough so that a chest tube T is inserted therein.

In addition, the body part 110 may have an outer skin 112 formed therein to surround an inner skin 111 that forms the insert guide hole 120, and a drug-receiving part S may be provided between the inner skin 111 and the outer skin 112.

Further, a drug injection part 130 may be formed on a rear side of the body part 110, and a drug discharge port 140 may be formed in the outer skin 112.

Moreover, the inner skin 111 may be formed of a ductile material, and the outer skin 112 may be formed of a rigid material.

Furthermore, a suture wing 150 may be formed at a rear end of the body part 110.

In addition, at least one fixing hole 151 may be formed in the suture wing 150.

Further, a kit K for inserting a chest tube and injecting a drug according to one embodiment of the present disclosure includes: a chest tube inserter 100 having an insert guide hole 120 formed through an inside of a body part 110 so that a chest tube T is inserted, wherein the body part 110 is formed in a double skin structure so that a drug-receiving part S is provided on an inside thereof; and a cutter 200 having a blade 210 formed at a front thereof and configured to incise a chest wall in a state of being inserted into the insert guide hole 120 of the chest tube inserter 100.

In addition, a cover pad 300 attached to a skin to cover the inserted chest tube inserter 100 may be further included.

Furthermore, a chest tube exposure hole 310 may be formed in the cover pad 300 at a location where the chest tube T passes through.

### Effects of the Disclosure

According to the chest tube inserter 100 for chest tube insertion and drug injection of the present disclosure, the chest tube can be inserted stably into the chest cavity as the chest tube is inserted through the insert guide hole after incising the chest wall to a minimum size and inserting a flat body part, and at the same time, the patient's pain can be reduced by minimizing the incision of the chest wall.

Further, the body part is formed in a double skin structure and is provided with a drug-receiving part, and thus, drugs can be supplied effectively so as to prevent inflammation from occurring at the incision site.

In addition, a suture wing is formed at the rear end of the body part and is fixed to the skin with a suture, and accordingly, the body part and the chest tube can be fixed stably so as not to move.

Furthermore, according to the kit K including the chest tube inserter of the present disclosure, the cutter having a blade formed at the front thereof makes an incision in a state of being inserted into the insert guide hole of the chest tube inserter, and thus, the incision site can be formed accurately and uniformly.

In addition, when the pointed blade moves forward and incises the chest wall, the chest tube inserter moves naturally and is positioned along the incision site, and therefore, the chest tube inserter can be inserted with the incision minimized, thereby reducing the patient's pain.

Further, the time point at which the chest wall is penetrated can be immediately identified through the patient's aspiration by moving the pointed blade of the cutter forward minutely and gradually, and thus, the blade can be prevented from being inserted excessively into the chest cavity, causing damage to the lungs or other organs.

### BRIEF DESCRIPTION OF THE DRAWINGS

FIG. 1 is a perspective view showing a chest tube inserter according to one embodiment of the present disclosure;

FIG. 2 is a cross-sectional view showing a state of use of the chest tube inserter according to one embodiment of the present disclosure;

FIG. 3 is a longitudinal-sectional view showing a state of use of the chest tube inserter according to one embodiment of the present disclosure;

FIG. 4 is a diagram of a state of use showing a state in which the chest tube inserter is fixed according to various embodiments of the present disclosure;

FIG. 5 is a perspective view showing the configuration of a kit according to one embodiment of the present disclosure;

FIG. 6 is a cross-sectional view showing a process in which a cutter and a chest tube inserter of the kit incise the chest wall according to one embodiment of the present disclosure;

FIG. 7 is a diagram of a state of use showing a state in which a cover pad is attached according to one embodiment of the present disclosure; and

FIG. 8 is a conceptual diagram showing, in a time series, the process of treating the incision site after removing the chest tube inserter of the present disclosure.

### DETAILED DESCRIPTION OF EMBODIMENTS

Hereinafter, embodiments of the present disclosure will be described in greater detail with reference to the accompanying drawings. The embodiments of the present disclosure may be modified in a variety of forms, and the scope of the present disclosure should not be construed as being limited to the embodiments below. The present embodiments are provided to more completely describe the present disclosure to those having ordinary knowledge in the art.

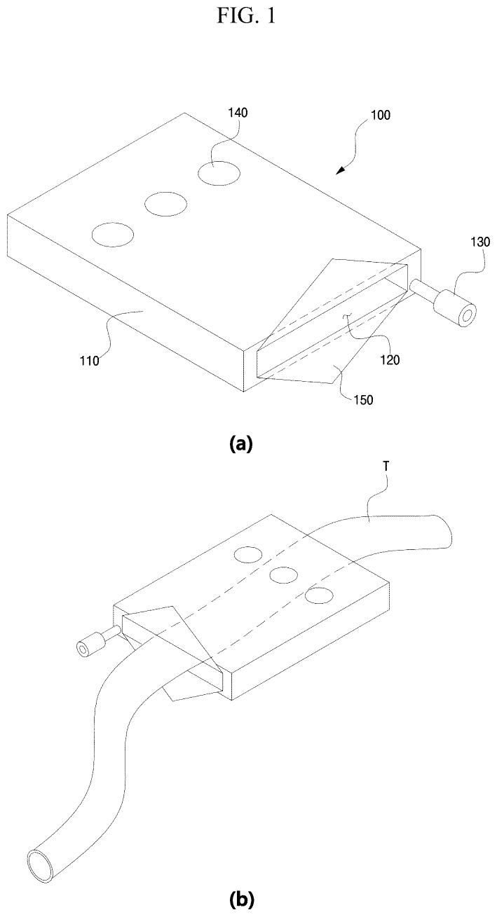

The present disclosure is proposed in such a way that a chest tube inserter 100 is provided to penetrate the chest wall so that a chest tube T can be easily inserted into the chest cavity in performing chest tube insertion, and as shown in FIG. 1, a body part 110 has an insert guide hole 120 formed on the inside thereof, thereby having the shape of a hollow tubular body.

In this case, “backward” is defined as the outward direction of the chest wall based on the direction in which the chest tube T is inserted, and “forward” is defined as the inward direction of the chest wall toward the chest cavity in the present disclosure.

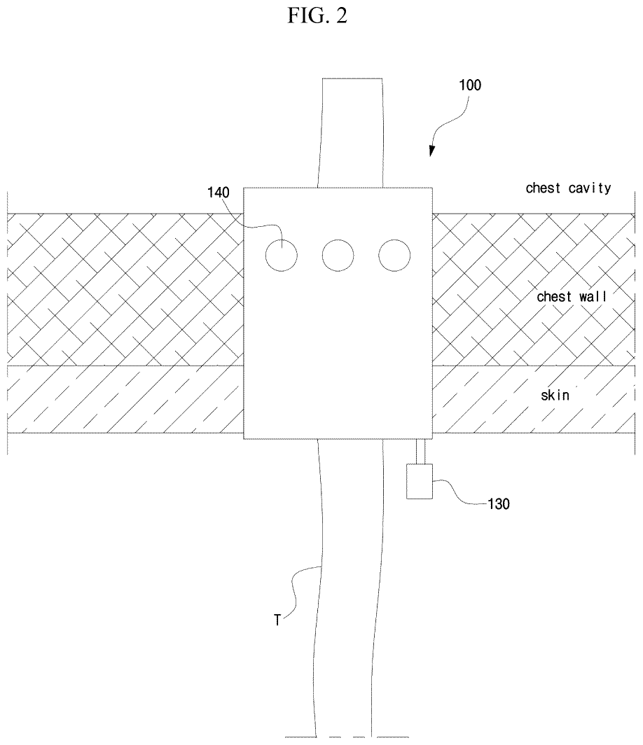

Further, the body part 110 is preferably formed to have a width W larger than a thickness D so as to facilitate insertion into the incision site, thereby having a flat shape as a whole, and as shown in FIG. 2, the chest tube T is inserted into the insert guide hole 120 of the chest tube inserter 100, and thus, the end is located inside the chest cavity.

As a result, the body part 110 in a flat form can be inserted effectively through the incision site formed transversely, the chest tube T can be inserted stably as the chest tube T is inserted into the chest tube inserter 100 fixed to penetrate the chest wall and at the same time, the incision of the chest wall can be minimized in the process, and the patient's pain can be reduced by preventing damage to surrounding tissues.

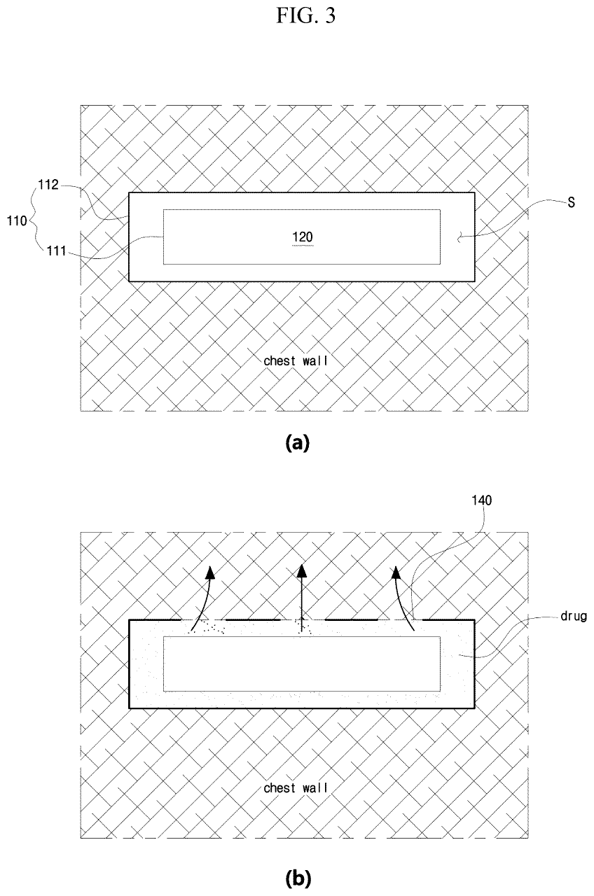

Further, since the body part 110 has a double skin structure as a whole as an outer skin 112 is formed to surround an inner skin 111 that forms the insert guide hole 120 as shown in FIG. 3, a drug-receiving part S is provided between the inner skin 111 and the outer skin 112.

In this case, a drug injection part 130 is formed on the rear side of the body part 110, and a drug discharge port 140 is formed in the outer skin 112, thereby continuously supplying a drug to the drug-receiving part S, and the drug injected into the drug-receiving part S can be stably supplied to the incision site of the chest wall through the drug discharge port 140, enabling the control of pain.

In addition, the inner skin 111 of the body part 110 is preferably made such that it is formed of a ductile material, thereby being deformed effectively according to the amount of drug injected into the drug-receiving part S, and the outer skin 112 is preferably formed of a rigid material so as not to deform its shape by external forces, allowing it to move stably in the process of being inserted into the incision site.

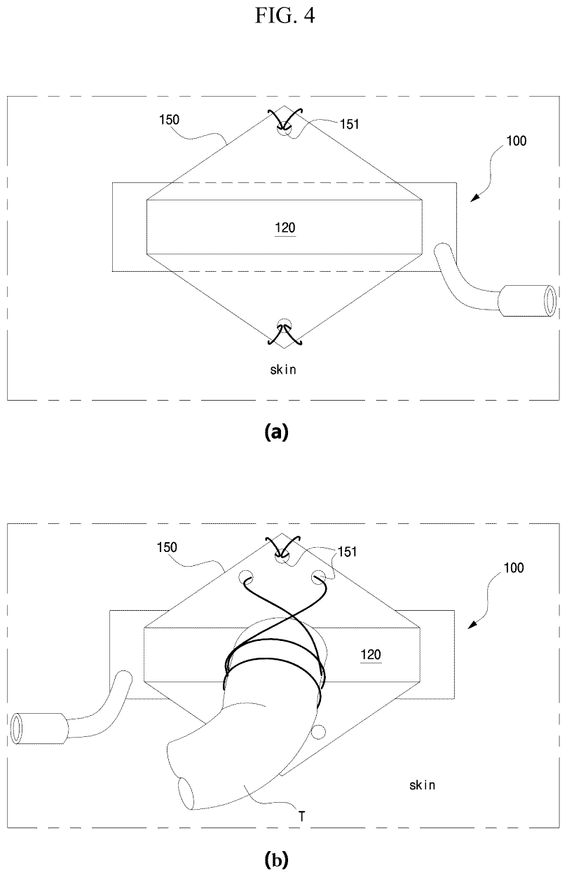

Further, a suture wing 150 may be additionally formed at the rear end of the body part 110 as shown in (a) of FIG. 4, and can be fixed with a suture while in contact with the skin adjacent to the incision site of the patient.

The suture wings 150 are preferably formed in a pair on the long side of the body part 110 so as to be stably fixed to the skin, and at least one fixing hole 151 may be formed in the suture wing 150, and thus, sutures can be sewn and fixed so as to pass through the fixing hole 151.

In addition, as shown in (b) of FIG. 4, the fixing holes 151 may be formed in plurality, some of which may be used to fix the suture wing 150 and the patient's skin, and some may be used to tie the chest tube T to the suture wing 150, thereby preventing the movement of the chest tube T and inducing stable fixation.

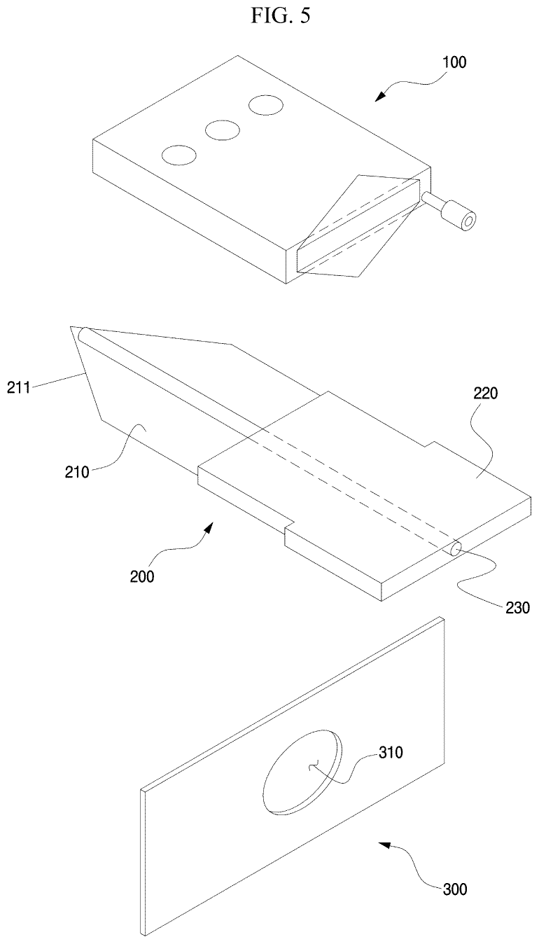

In the following, a kit K including the foregoing chest tube inserter 100 for chest tube insertion and drug injection will be described. FIG. 5 shows the configuration of a kit according to one embodiment of the present disclosure, and the kit K of the present disclosure may include the chest tube inserter 100, a cutter 200, and a cover pad 300.

The cutter 200 has a pointed blade 210 formed at the front thereof, and as it moves forward, the pointed tip of the blade 210 incises the chest wall. In this case, a guide body 220 may be formed at the rear of the blade 210 so that the cutter 200 can move stably along the insert guide hole 120 of the chest tube inserter 100.

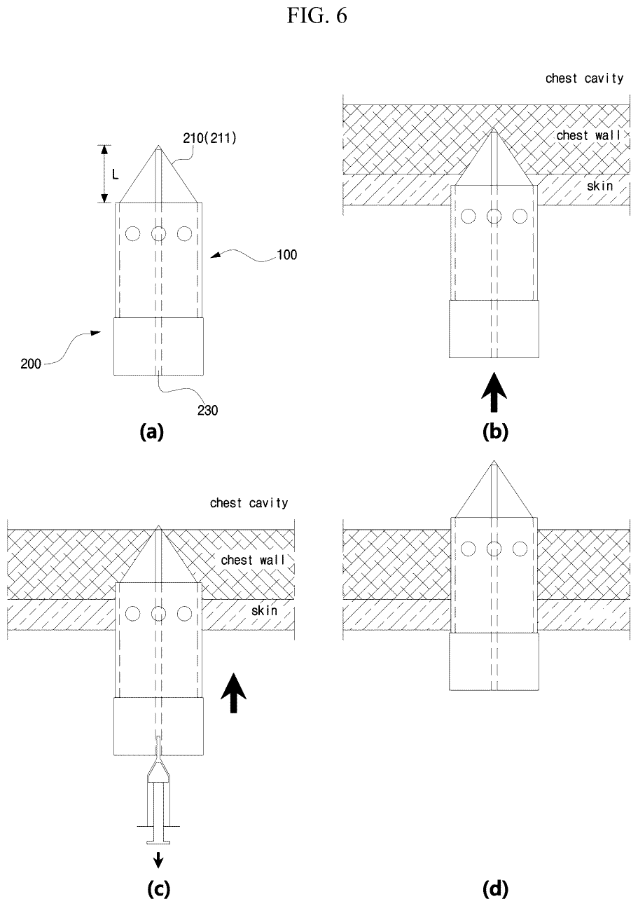

FIG. 6 shows a process in which the chest wall is incised by the cutter 200 inserted into and moved in the chest tube inserter 100 of the kit K according to one embodiment of the present disclosure, and the cutter 200 is inserted into the insert guide hole 120 of the chest tube inserter 100 ((a) of FIG. 6), and the cutter 200 is gradually moved forward ((b) of FIG. 6). At this time, the pointed blade 210 of the cutter 200 moves forward while incising the chest wall, and the chest tube inserter 100 also moves forward along the incised site.

Further, the pointed blade 210 of the cutter 200 is gradually moved forward and is made to enter the inside of the chest wall. In this case, a needle hole 230 is formed in the cutter 200 so as to pass through the pointed blade 210, and thus, air or liquid present inside the chest cavity can be sucked in with a syringe, allowing for instantly checking whether the blade 210 has entered the inside of the chest wall well ((c) of FIG. 6). Thereby, the blade 210 can be prevented from being inserted excessively into the chest cavity, causing damage to the lungs or other organs.

Then, when an incision site is formed with a sufficient width by moving the cutter 200 further forward by the length L of the pointed tip 211 of the blade 210, the chest tube inserter 100 is also placed in the right position so as to penetrate the chest wall ((d) of FIG. 6). In this case, the length L of the pointed tip 211 may preferably be formed in a length that does not cause damage to organs, thereby ensuring safety while inserting the chest tube inserter 100.

Therefore, according to the kit K including the chest tube inserter of the present disclosure, since the cutter 200 with the blade 210 formed therein is moved stably along the insert guide hole 120 of the chest tube inserter 100, the incision site can be formed accurately and uniformly. In addition, when the pointed blade 210 moves forward and incises the chest wall, the chest tube inserter 100 can be inserted with the incision minimized as the chest tube inserter 100 moves along the incision site, and thus, uniform and accurate treatment is possible regardless of the skill level of the doctor, and the patient's pain can also be reduced.

In this case, the chest tube inserter 100 may be formed to move forward naturally together with the movement of the cutter 200, but alternatively, it may be formed such that the cutter 200 moves forward and backward independently along the insert guide hole 120 of the chest tube inserter 100, and the chest tube inserter 100 also moves independently regardless of the movement of the cutter (200), as a matter of course.

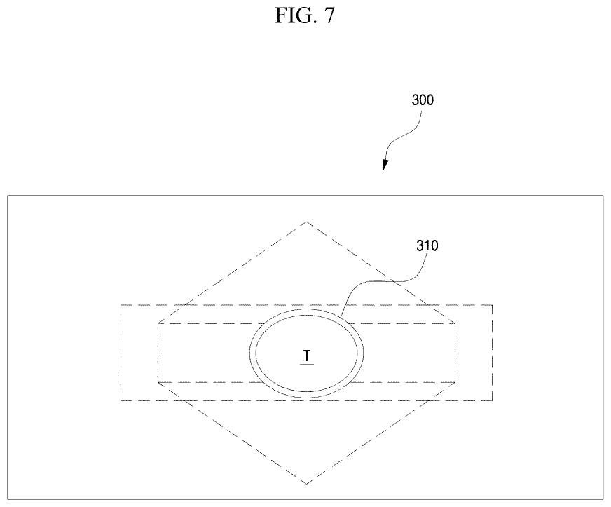

Further, as shown in FIG. 7, the kit K including the chest tube inserter of the present disclosure may be treated so as not to be exposed to external contamination sources by the cover pad 300 that is attached to the skin while the chest tube inserter 100 is inserted to penetrate the chest wall. In addition, the cover pad 300 functions to stably fix the chest tube inserter 100 and the chest tube T to the patient's body so that they do not move.

However, since the chest tube T must be exposed to the outside by passing through the cover pad 300, a chest tube exposure hole 310 is formed in the cover pad 300 at the location where the chest tube T passes through.

The process of removing the chest tube T after the chest tube inserter 100 of the present disclosure is positioned to penetrate the chest wall with the chest tube T provided therein proceeds in the reverse sequence of the inserting process. First, the chest tube T and the cover pad 300 are removed in sequence. Then, the sutures of the suture wings 150 exposed behind the chest tube inserter 100 are removed, and the chest tube inserter 100 is pulled backward and removed.

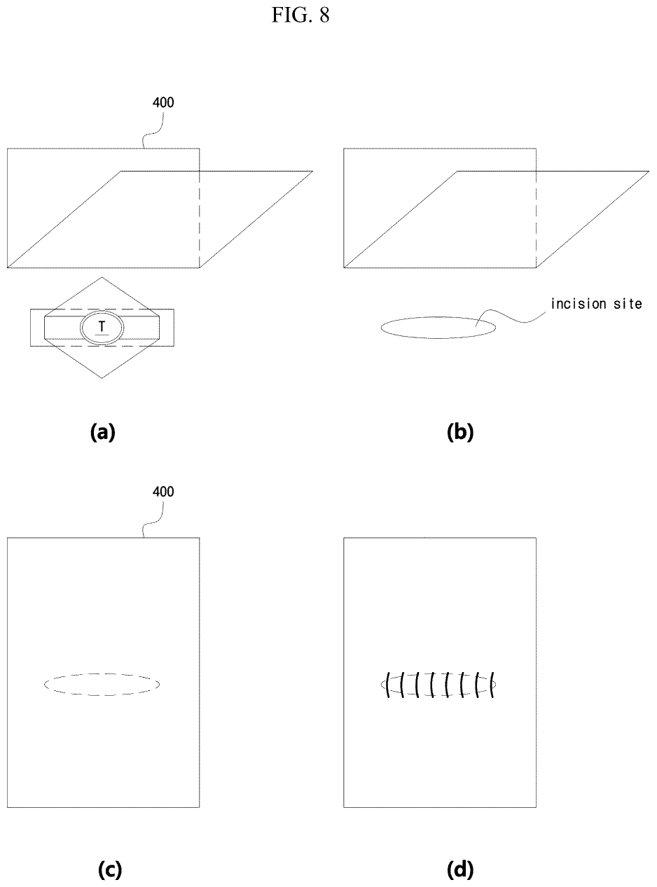

Further, FIG. 8 is a conceptual diagram showing, in a time series, the process of treating the incision site after removing the chest tube inserter 100 of the present disclosure. In a state where a tape 400 is first attached temporarily to one side before removing the chest tube inserter 100 described above ((a) of FIG. 8), the chest tube inserter 100 is removed ((b) of FIG. 8), and the tape 400 is attached completely so as to cover the incision site ((c) of FIG. 8). Next, the incision site is treated to be closed using sutures or stapler on top of the tape 400 ((d) of FIG. 8).

The descriptions have been provided above with a focus on the embodiments of the chest tube inserter 100 for chest tube insertion and drug injection and the kit K including the same of the present disclosure, but those having ordinary skill in the art will be able to implement the present disclosure by modifying and changing it in various ways through addition, change, deletion, or the like of components within the scope that does not depart from the spirit of the present disclosure set forth in the claims, which will also be considered to fall within the scope of the present disclosure.

Furthermore, in describing the embodiments of the present disclosure, if it has been determined that specific descriptions of known functions or components may unnecessarily obscure the gist of the present disclosure, the detailed descriptions have been omitted. In addition, the terms used are terms defined in view of the functions in the embodiments of the present disclosure, and may vary depending on the intention or relationship of the user or operator, etc. Consequently, the definitions should be made based on the contents throughout the present specification. Therefore, the detailed description of the disclosure above is not intended to limit the present disclosure to the disclosed embodiments, and the appended claims should be construed to include other embodiments.

### INDUSTRIAL APPLICABILITY

The chest tube inserter for chest tube insertion and drug injection and the kit including the same of the present disclosure allow easy insertion of the chest tube without pain in inserting it so as to penetrate the chest wall, and facilitate drug injection as the body part is formed in a double skin structure.

1. A chest tube inserter (100) for inserting a chest tube, which is provided to penetrate a chest wall,
wherein a body part (110) of the chest tube inserter (100) has an insert guide hole (120) formed therethrough so that a chest tube (T) is inserted therein.

2. The chest tube inserter of claim 1, wherein the body part (110) has an outer skin (112) formed therein to surround an inner skin (111) that forms the insert guide hole (120), and a drug-receiving part(S) is provided between the inner skin (111) and the outer skin (112).

3. The chest tube inserter of claim 2, wherein a drug injection part (130) is formed on a rear side of the body part (110), and a drug discharge port (140) is formed in the outer skin (112).

4. The chest tube inserter of claim 2, wherein the inner skin (111) is formed of a ductile material, and the outer skin (112) is formed of a rigid material.

5. The chest tube inserter of claim 1, wherein a suture wing (150) is formed at a rear end of the body part (110).

6. The chest tube inserter of claim 5, wherein at least one fixing hole (151) is formed in the suture wing (150).

7. A kit for inserting a chest tube and injecting a drug, comprising:
a chest tube inserter (100) having an insert guide hole (120) formed through an inside of a body part (110) so that a chest tube (T) is inserted, wherein the body part (110) is formed in a double skin structure so that a drug-receiving part(S) is provided on an inside thereof; and
a cutter (200) having a blade (210) formed at a front thereof and configured to incise a chest wall in a state of being inserted into the insert guide hole (120) of the chest tube inserter (100).

8. The kit for inserting a chest tube and injecting a drug of claim 7, further comprising:
a cover pad (300) attached to a skin to cover the inserted chest tube inserter (100).

9. The kit for inserting a chest tube and injecting a drug of claim 8, wherein a chest tube exposure hole (310) is formed in the cover pad (300) at a location where the chest tube (T) passes through.
